(19) United States
(12) Patent Application Publication
(10) Pub. No.: US 2025/0304743 A1
(43) Pub. Date: Oct. 2, 2025

(54) CHARACTERIZATION AND CHANGE OF POLYHYDROXYALKANOATE (PHA) CRYSTALLOGRAPHIC PHASE

(71) Applicant: HORIBA Instruments Incorporated, Irvine, CA (US)

(72) Inventor: Fran Adar, Irvine, CA (US)

(21) Appl. No.: 18/623,559

(22) Filed: Apr. 1, 2024

(51) Int. Cl. C08G 63/06; G01N 21/31; G16C 20/10

(52) U.S. Cl. CPC C08G 63/06; G01N 21/31; G16C 20/10; G01N 2201/06113

(57) ABSTRACT

A composition and/or processing conditions of a development sample of polyhydroxyalkanoate can be changed based on data gathered from techniques described herein to alter a crystallographic property of the development sample in an attempt to produce non-centrosymmetric β crystallographic structure in the development sample with stable piezoelectric properties. Such would have use in a variety of applications.

### TECHNICAL FIELD

This disclosure relates to polymer science.

### BACKGROUND

Polymer applications can be characterized by their dynamic progression and increasing relevance in various sectors such as technology, healthcare, and sustainability. The field is driven by the demand for materials that not only meet specific performance criteria but are also environmentally sustainable and exhibit novel functionalities. One of the major trends is the development of biodegradable and bio-based polymers aimed at minimizing environmental impacts by breaking down naturally or being derived from renewable resources.

There is also growing interest in smart and responsive polymers, which are designed to alter their properties in response to external stimuli like temperature, pressure, light, humidity, or electrical fields. Such materials find applications in diverse areas, including drug delivery systems that react to physiological conditions and self-healing materials capable of responding to mechanical damage. The incorporation of nanoparticles or the blending of different polymers to form nanocomposites and polymer blends is another avenue being explored to enhance material properties such as strength, thermal stability, and conductivity, catering to the needs of advanced electronics, packaging, and aerospace sectors.

The advent of 3D printing and additive manufacturing with polymers has brought about a revolution in prototyping and manufacturing, enabling the creation of structures with complex geometries.

In terms of engineering techniques, the field employs various techniques to facilitate control over polymer structure, enabling the synthesis of complex architectures tailored for specific functions. Chemical modification of polymers post-polymerization is another strategy to introduce new functional groups or create cross-links between chains, thereby altering material properties. Blending and compounding polymers with additives or other polymers allows for the customization of properties to achieve desired mechanical, thermal, or barrier characteristics.

Processing techniques such as extrusion, injection molding, spinning, and film blowing also play a role in shaping polymers into final products. Advances in these areas aim at enhancing efficiency, minimizing waste, and improving the properties of materials. Additionally, the use of advanced analytical techniques alongside computational modeling and simulation is useful for understanding the relationships between polymer structure and its properties, aiding in the prediction of material behavior and the design of polymers with targeted characteristics.

### SUMMARY

A method includes illuminating a test sample of polyhydroxyalkanoate (PHA) with monochromatic laser light having a predefined wavelength such that the test sample scatters the monochromatic laser light to generate scattered light at multiple wavelengths, measuring wavenumber shift and intensity of the scattered light during or after crystallization as a function of time or a function of sample manipulation (e.g., stretching), and confirming presence of high energy aliphatic carbon-hydrogen (C—H) doublet bands defined by the wavenumber shift and intensity, indicative of a non-centrosymmetric phase in the test sample, and associated with components of a carbonyl band that are indicative of different phases in the test sample. The method also includes searching for correlations between the C—H doublet bands and the components of the carbonyl band and changing a composition or processing conditions of a development sample of PHA based on presence or absence of the correlations to alter a crystallographic property of the development sample in an attempt to produce non-centrosymmetric β crystallographic structure in the development sample with stable piezoelectric properties.

### BRIEF DESCRIPTION OF THE DRAWINGS

FIG. 1 is a plot of the first and last Raman spectra of 13.8% polyhydroxybutyrate-hydroxyhexanoate (PHBHx) recorded during isothermal crystallization, including intensity trends for the low frequency phonon band, the carbonyl band, and the high frequency C—H bands.

FIG. 2 is the asynchronous plot of C—H bands resulting from two-dimensional correlation analysis of Raman measurements of PHBHx undergoing isothermal crystallization during the middle period of crystallization.

FIG. 3 shows the hetero-mode correlation plots of C—H versus carbon-oxygen double bond (>C═O) vibrational modes.

FIG. 4 is a schematic representation of the molecular structure of the α helix and β sheet forms of polyhydroxybutyrate (PHB) showing the net polarization between the methylene and carbonyl groups.

FIG. 5 is a flow chart of a method for characterizing and changing PHBHx piezoelectric properties.

### DETAILED DESCRIPTION

Embodiments are described herein. It is to be understood, however, that the disclosed embodiments are merely examples and other embodiments may take various and alternative forms. The figures are not necessarily to scale. Some features could be exaggerated or minimized to show details of particular components. Therefore, specific structural and functional details disclosed herein are not to be interpreted as limiting, but merely as a representative basis for teaching one skilled in the art.

Various features illustrated and described with reference to any one of the figures may be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

PHBHx is a biodegradable polymer that belongs to the family of polyhydroxyalkanoates (PHAs). PHAs are a class of polyesters naturally produced by bacterial fermentation under conditions of nutrient imbalance. PHBHx is interesting due to its biodegradability and biocompatibility, which makes it a promising material for a wide range of applications, especially in areas where environmental protection is a concern.

PHBHx is a copolymer composed of two monomers: hydroxybutyrate (HB) and hydroxyhexanoate (HHx). The HB units are typically synthesized from acetyl-CoA through a bacterial fermentation process by a variety of bacteria, such as Cupriavidus necator. The bacteria accumulate PHAs, including PHBHx, as intracellular granules under conditions of nutrient limitation with excess carbon sources. The molecular weight of PHBHx can vary depending on the fermentation process and bacterial strain used.

The monomers are linked together through ester bonds, formed between the hydroxyl group of one monomer and the carboxyl group of the next. This results in a long-chain polyester. These ester linkages are susceptible to hydrolytic degradation, contributing to its biodegradability. Both HB and HHx units are aliphatic, consisting of linear or branched carbon chains without aromatic rings or olefinic bonds. This also contributes to its biodegradability.

The crystallinity (i.e., the extent and way polymer chains organize into ordered structures) of PHBHx is influenced by the ratio of polyhydroxybutyrate (PHB) to HHx. Higher PHB content generally increases crystallinity, rigidity, and melting point, while higher HHx content tends to make the polymer more amorphous and lowers the melting point which, in turn, makes the material more useful in industrial applications. PHBHx thus exhibits a balance between stiffness and flexibility, which can be tuned by varying the PHB/HHx ratio. This tunability allows for a wide range of applications, from rigid materials to more elastic ones.

PHBHx, like other polymers, forms a semi-crystalline structure as alluded to above, where parts of the polymer are crystalline, and parts are amorphous. The crystalline regions are formed by the orderly packing of polymer chains, typically driven by the PHB content. In areas where the polymer chains in the solid are randomly arranged and not ordered, PHBHx exhibits an amorphous phase. The amorphous regions contribute to the material's flexibility and impact resistance. A higher proportion of HHx typically increases the amorphous character of PHBHx as mentioned earlier, due to the larger, bulkier side chains of HHx disrupting the regular packing of the polymer chains. The degree of crystallinity in PHBHx affects its physical properties like tensile strength, modulus, and thermal resistance. In some conditions, particularly with a high content of PHB and appropriate thermal processing, PHBHx can exhibit a surprisingly high crystallinity.

The composition of PHBHx (ratio of HB to HHx) will influence its glass transition temperature, the temperature at which the polymer transitions from a hard and glassy state to a soft and rubbery state. Upon further heating, PHBHx will reach a melting point where the crystalline regions melt. The melting point is a factor for processing the polymer and is influenced by the degree of crystallinity.

PHBHx has applications in various fields, including packaging, agricultural films, and biomedical applications such as sutures and drug delivery systems. Its biocompatibility and biodegradability make it particularly suitable for certain applications. Ongoing research is focused on, among other things, finding new applications for this material.

Raman spectroscopy, a spectroscopic technique based on the Raman effect, may be used for such research, and involves the inelastic scattering of monochromatic light from a laser source. When light interacts with molecular vibrations within a sample, the energy of the photons is shifted up or down. This shift provides information about the vibrational modes in the system, which can be directly related to the molecular and crystalline structure of the sample. Sample preparation can be minimal in Raman spectroscopy, and samples can often be analyzed in their natural state, whether solid, liquid, or gas.

The component of a Raman system that excites the spectrum is a laser source. The laser provides, in certain arrangements, polarized monochromatic light that interacts with the sample. The choice of laser wavelength is determined by the sample's optical properties and the desired information. The laser beam is directed and focused onto the sample using lenses and mirrors. The sample is typically placed in a holder that can be precisely positioned and manipulated; often a microscope is the sampling device because of its ease of use and high efficiency, especially for small samples. After interaction with the sample, scattered light is collected. This collection is typically done with mirrors and lenses that focus the scattered light onto a spectrograph. The spectrograph disperses the light and focusses it onto a multichannel detector. The detector captures the spectrum, and a computer with spectroscopic software provides a plot of intensity versus frequency shift (Raman shift). This same computer system with specialized software is also used to control the instrument and process the spectra.

The Raman shift is the difference in energy between the incident laser light and the scattered light. It is expressed in frequency units, usually wavenumbers (cm⁻¹), which are inversely proportional to wavelength. The shift occurs due to the inelastic scattering of photons, where energy is either gained or lost by the photons upon interaction with the sample's molecular vibrations.

The peaks in the spectrum correspond to the vibrational modes of the molecules and/or crystals in the sample. The intensity of the scattered light at each Raman shift is plotted on the y-axis. The intensity of a Raman peak is related to the polarizability of the molecular bonds and the concentration of the corresponding molecular species in the sample. Each peak in the Raman spectrum corresponds to a specific vibrational mode of the molecules and crystals in the sample. The position (wavenumber) of a peak is determined by the energy difference between the incident and scattered photons, which is equal to the vibrational energy of a particular molecular bond or group.

The regions of the spectrum where most of the characteristic Raman peaks appear are called the “fingerprint and carbon-hydrogen stretching regions.” These regions are particularly useful for identifying specific compounds or functional groups within a molecule. By analyzing the positions and intensities of the peaks in a Raman spectrum, one can infer various aspects of the structure of the sample. This includes information about bond types, symmetry, molecular conformations, and crystal packing. There is also information in the low frequency region which includes molecular phonons in crystals; phonons represent beating of the molecular units against each other in the crystallographic unit cell.

When examining PHBHx via Raman spectroscopy, specific scattering features associated with stretching vibrations of aliphatic carbon-hydrogen (C—H) bonds provide information about its structure. These stretches arise from the methine (CH), methylene (—CH₂—), and methyl (—CH₃) groups present in the polymer's backbone and side chains. The C—H₂ and C—H₃ bonds in aliphatic hydrocarbons exhibit characteristic vibrational modes: symmetric stretch when the respective two or three hydrogen atoms bonded to the same carbon atom move in phase and asymmetric stretch when these hydrogen atoms move in opposite directions. The aliphatic nature of these groups is reflected in their vibrational stretching frequencies, observed usually in the range of 2800 cm⁻¹ to 2960 cm⁻¹ in vibrational (infrared and Raman) spectroscopy. These are characteristic of C—H bonds in saturated hydrocarbons.

In spectroscopy, the C—H vibrations typically manifest as bands in the high-energy region of the spectrum. The intensity of these C—H bands in a spectrum is related to the concentration of the aliphatic C—H bonds in the sample and the nature of their vibrational modes. Higher intensity suggests a higher concentration or more pronounced vibrational activity. Crystalline PHAs show an unusual pair of bands between 2990 cm⁻¹ and 3010 cm⁻¹ which have been ascribed to a strong interaction between the carbonyl in one chain and a methyl group in the second chain in a unit cell.

The carbonyl (C═O) stretching region in PHBHx is associated with the ester linkage in the polymer chain. While the carbonyl group itself is not aliphatic, it is part of the overall aliphatic polyester structure of PHBHx. The C—O stretching vibration of esters is usually observed between 1720 cm⁻¹ and 1750 cm⁻¹ in vibrational spectroscopy (Raman and infrared).

To analyze PHBHx via Raman spectroscopy, a sample was cast as a film. A small piece was cut off and mounted on a stainless-steel microscope slide. The slide was mounted on a hotplate, and when the film melted, it was moved into a cup of liquid nitrogen and quenched. The stainless-steel slide with the sample was mounted on the microscope stage of a Raman microscope equipped with a laser, spectrograph, and detector capable of at least 0.3 cm⁻¹ to 0.5 cm⁻¹ resolution and 1 cm⁻¹ accuracy in spectral shift.

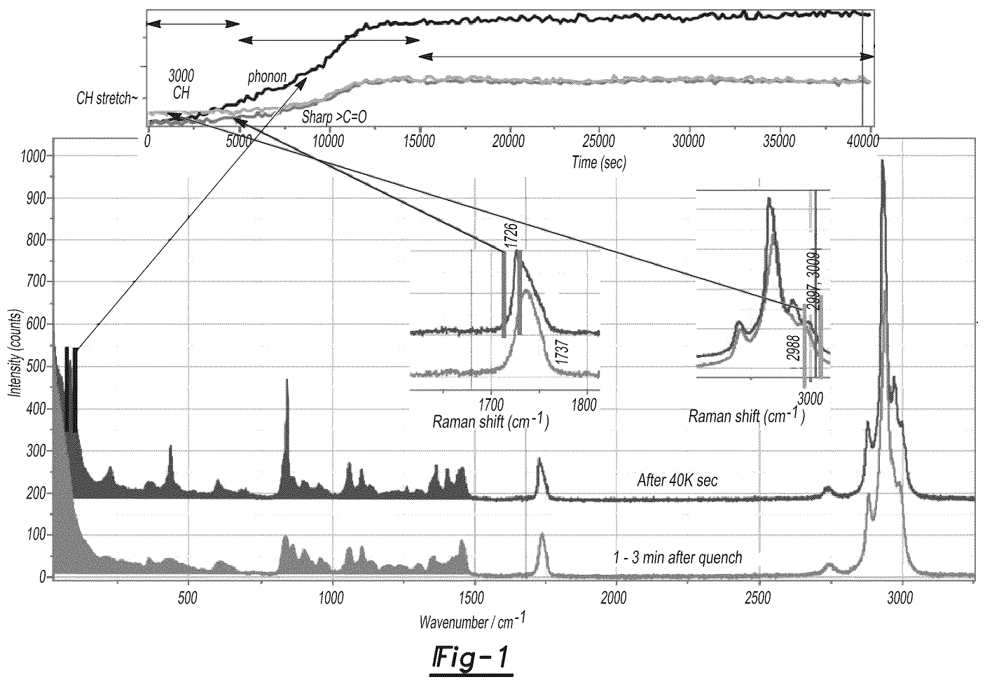

The spectrum was scanned, over a period of at least 10 hours, between 30 cm⁻¹ to 3200 cm⁻¹, which is the end of the Stokes spectrum of a hydrocarbon with no nitrogen or oxygen. FIG. 1 shows the spectra taken at the beginning and the end of the 10-hour period. Three specific bands were chosen to indicate the sample's evolution. Because the Raman effect is produced by a single beam measurement as described above, appropriate normalization of spectra is required; the spectra were prepared for further analysis using baseline-subtraction and then normalization to the unit vector.

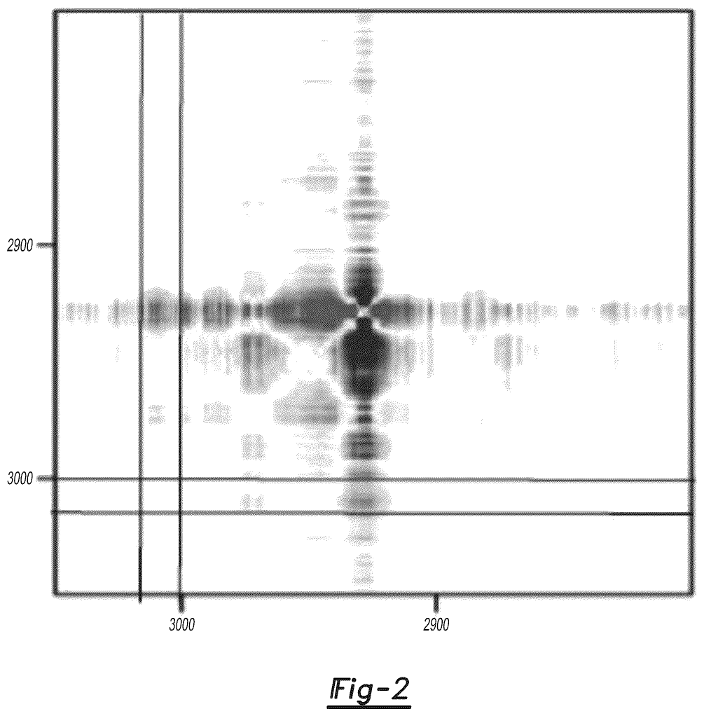

Two-dimensional correlation analysis was then performed. Both homo-mode and hetero-mode correlation plots (synchronous and asynchronous) for the carbonyl and C—H bands were developed, representing periods of the early phase, the middle phase, and the final phase of crystallization. The various features in the carbonyl and C—H regions were followed during the different times from which it was possible to assemble a sequence of changes as the crystallization proceeds. In particular, the asynchronous plot of the C—H region during the intermediate phase (FIG. 2) indicated the absence of bands near 3000 cm⁻¹ and 3010 cm⁻¹, indicating these bands appear simultaneously and thus can be interpreted as evidence for correlation field splitting in the crystal, with the implication there cannot be a center of symmetry.

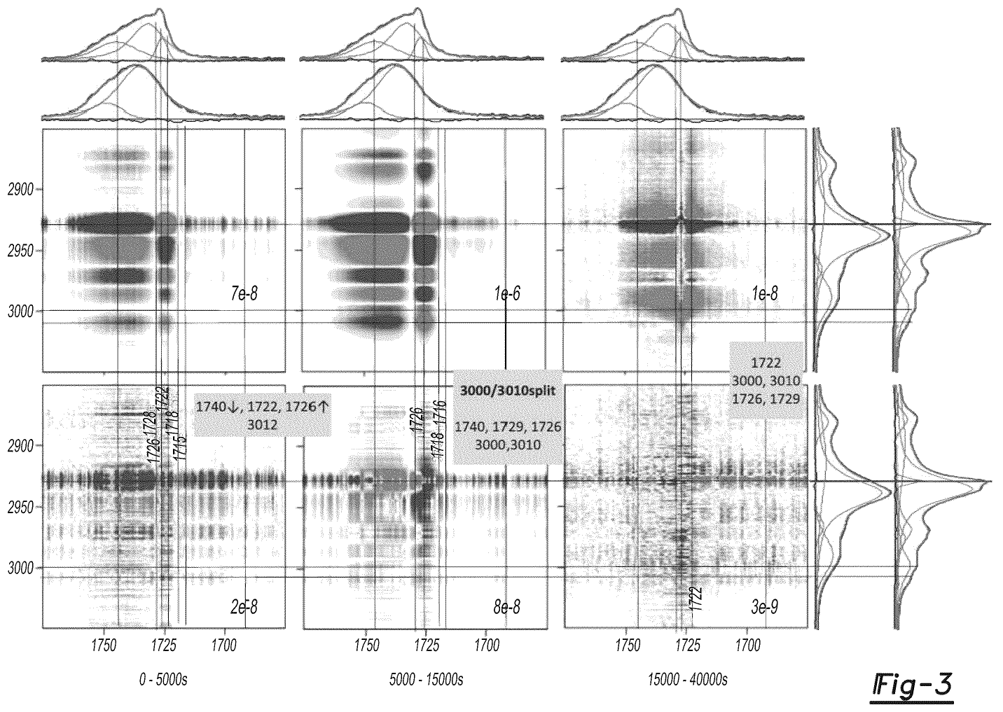

In the hetero-mode, correlation plots for the C—H bands versus both the carbonyl bands (FIG. 3) and the low frequency phonon modes were generated. It was in the middle plots that the C—H band most clearly split into points at ˜3000 cm⁻¹ and ˜3010 cm⁻¹ in both the synchronous and asynchronous plots and the two bands appeared simultaneously (FIG. 2).

FIG. 2 shows an asynchronous plot in the C—H region for the middle period (representing 5000 sec to 15000 sec). At the intersection of the lines at 3000 cm⁻¹ and 3010 cm⁻¹, there is no intensity-indicating the bands at these positions appeared simultaneously. These two bands are a correlated pair. That is, they represent the C—H stretch in the two molecular chains in the crystal, which because of the correlation field in the crystal they interact and split apart; their simultaneous appearance in the Raman spectra indicate there cannot be a center of symmetry.

The inventor has recently realized that if the X-ray diffraction centrosymmetric structure that has been believed by the PHBHx community was accurate, it would not allow for both bands to be observed simultaneously via Raman spectroscopy in a crystal with centrosymmetric structure. The observation of the correlation field splitting thus precludes a centrosymmetric structure. Indeed, certain X-ray diffraction literature has noted the dearth of X-ray diffraction spots which can explain why the structure has been misassigned. There are also some observations in the early literature for a weak piezoelectric effect in the usual a phase under certain conditions.

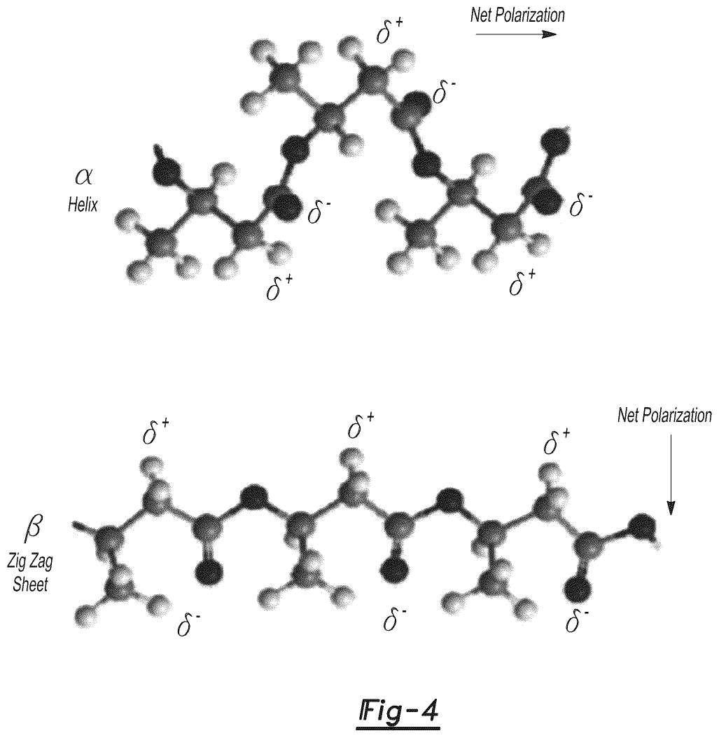

The piezoelectric effect requires a material to lack a center of symmetry in its crystal lattice (non-centrosymmetric phase). The common crystal form of PHBHx is not typically known for its piezoelectric properties. FIG. 4, however, indicates how the α helix can support a polarization, although it would clearly be weaker than that of the planar zig zag β form, and it would be aligned on a different axis. To the extent that it is believed in the art that PHAs can have a piezoelectric phase, that phase is generally thought to be unstable. The inventor nevertheless connects the correlated pair behavior noted above to a weak piezoelectric phase that may be compatible with subsequent formation of the β phase, which is known to be more highly piezoelectric. As a result, Raman measurements as described in the next paragraph can be used to aid in the engineering of PHBHx, and PHAs more generally, and to assist in determining conditions to stabilize this phase. Note that all PHA measurements in the crystalline form to date have shown two lines in the high C—H region.

Recent publications show clear markers for the formation of the beta phase in the carbonyl band in the Raman spectrum by correlating with X-ray diffraction. They show that the >C═O in the crystalline a phase appears between 1720 cm⁻¹ and 1725 cm⁻¹, in the amorphous phase near 1746 cm⁻¹, and in the β phase near 1740 cm⁻¹. In the asynchronous plot of the middle period in FIG. 3, one can also see there are multiple points of intensity in the carbonyl region.

Engineering the properties of PHBHx to alter its polymorphic crystalline phase and other aspects of its molecular structure may involve adjusting the nature of the second monomeric unit, adjusting the concentration ratio of its monomeric units, controlling the molecular weight, incorporating various additives, and fine-tuning the processing conditions, especially applying strain. As mentioned above, increasing the content of PHB tends to enhance the polymer's degree of crystallinity, as PHB units pack more efficiently into a crystalline structure, resulting in increased stiffness, higher melting temperature, and slower degradation rate; whereas a higher concentration of HHx or other monomers generally reduces crystallinity due to the larger side chains disrupting the regular packing of polymer chains, leading to materials that are more flexible, possess lower melting temperatures, and are less brittle.

The integration of additives may play a role in modulating the properties of PHBHx. Nucleating agents, for example, provide nucleation sites that facilitate crystal formation, thereby increasing the crystallization rate and degree of crystallinity, which enables control of mechanical properties. Plasticizers can be added to decrease the interaction between polymer chains, reducing the material's glass transition temperature and increasing its flexibility. The inclusion of fillers or reinforcements, such as inorganic fillers or organic fibers, can further enhance the mechanical properties and modify the properties of composites.

The processing conditions, including temperature, pressure/strain, and cooling rate, also affect the final properties of PHBHx. The temperature at which PHBHx is processed affects its crystallinity and piezoelectricity, with higher processing temperatures potentially leading to a more crystalline structure, though excessive heat may cause thermal degradation. The cooling rate from the melt influences the degree of crystallinity as well. Rapid cooling tends to result in a less ordered, amorphous state, whereas slow cooling allows more time for the chains to arrange into a crystalline structure. Additionally, annealing by heating to a temperature below its melting point and then slowly cooling can increase its degree of crystallinity, allowing the polymer chains to rearrange into a more ordered structure. And applying stress during heating and/or cooling can introduce orientation and can affect the amount and/or type of crystallinity (a or B) and the size of the crystallites.

By systematically adjusting these parameters and monitoring corresponding effects on crystallographic properties via the methods described herein, especially using two-dimensional correlation analysis to identify the presence of carbonyl and other bands (including the C—H bands, which have been seen to shift in two-dimensional correlation analysis plots for, as yet, unknown reasons) indicative of the β piezoelectric phase, it is possible to engineer PHA into a stable piezoelectric phase, which would have commercial applications in a variety of applications.

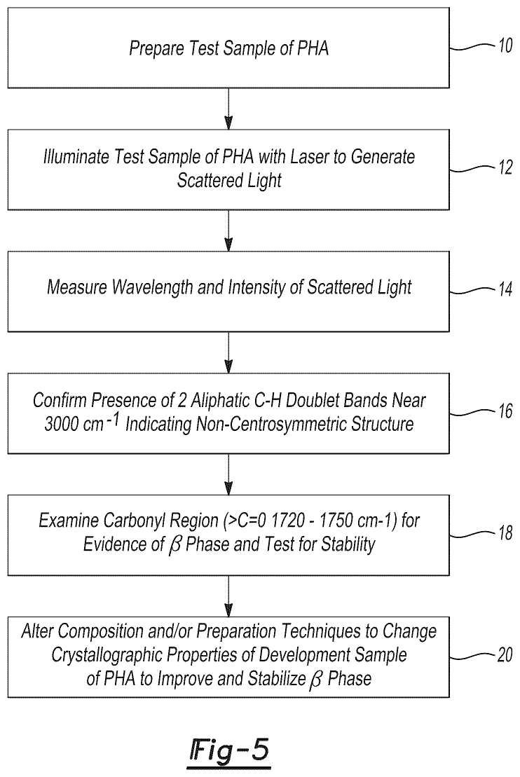

Referring to FIG. 5, a test sample of PHA is prepared for spectroscopic examination at operation 10 as described above. At operation 12, the test sample is illuminated with polarized monochromatic laser light having a predefined wavelength such that the test sample scatters the polarized monochromatic laser light to generate scattered light. At operation 14, wavelength and intensity of the scattered light is measured. At operation 16, high energy aliphatic C—H bands appearing in the intensity are correlated to characterize the molecular structure of the test sample to confirm the non-centrosymmetric phase appears. At operation 18, the carbonyl region is examined for evidence of the β phase. This can include searching for correlations between the C—H doublet bands and components of the carbonyl band. At operation 20, the composition or processing conditions of a development sample of PHA is changed based on presence or absence of the correlations. This change is made to alter crystallographic properties of the development sample in an attempt to produce non-centrosymmetric β crystallographic structure in the development sample with stable piezoelectric properties. These operations may then be repeated as desired.

As the composition and processing conditions are adjusted by the polymer engineer to identify how to prepare a piezoelectric material, Raman spectra are collected and analyzed for crystallographic behavior. The two-dimensional correlation algorithm will be useful in determining in what order molecular functional groups are moving into which crystalline phase, and most importantly, whether the >C—O bands indicate which phase is appearing.

As suggested above, the techniques contemplated here are applicable to PHAs. The fundamental structure of PHAs is that of a polyester, consisting of repeating ester (—COO—) linkages in their backbone. This ester linkage is formed through the condensation of hydroxybutyrate (BH) monomers, where the hydroxyl group of one monomer reacts with the carboxyl group of another, releasing water and forming the ester bond. Given the structural similarities among the members of the PHA family, techniques effective for PHBHx can of course be adapted to other PHAs.

The algorithms, methods, or processes disclosed herein can be deliverable to or implemented by a computer, controller, or processing device, which can include any dedicated electronic control unit or programmable electronic control unit. Similarly, the algorithms, methods, or processes can be stored as data and instructions executable by a computer or controller in many forms including, but not limited to, information permanently stored on non-writable storage media such as read only memory devices and information alterably stored on writeable storage media such as compact discs, random access memory devices, or other magnetic and optical media. The algorithms, methods, or processes can also be implemented in software executable objects. Alternatively, the algorithms, methods, or processes can be embodied in whole or in part using suitable hardware components, such as application specific integrated circuits, field-programmable gate arrays, state machines, or other hardware components or devices, or a combination of firmware, hardware, and software components.

While exemplary embodiments are described above, it is not intended that these embodiments describe all possible forms encompassed by the claims. The words used in the specification are words of description rather than limitation, and it is understood that various changes may be made without departing from the spirit and scope of these disclosed materials.

As previously described, the features of various embodiments may be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics may be compromised to achieve desired overall system attributes, which depend on the specific application and implementation. As such, embodiments described as less desirable than other embodiments or prior art implementations with respect to one or more characteristics are not outside the scope of the disclosure and may be desirable for particular applications.

What is claimed is:

1. A method comprising:
illuminating a test sample of polyhydroxyalkanoate (PHA) with monochromatic laser light having a predefined wavelength such that the test sample scatters the monochromatic laser light to generate scattered light at multiple wavelengths;
measuring wavenumber shift and intensity of the scattered light during or after crystallization as a function of time or a function of sample manipulation;
confirming presence of high energy aliphatic carbon-hydrogen (C—H) doublet bands defined by the wavenumber shift and intensity, indicative of a non-centrosymmetric phase in the test sample, and associated with components of a carbonyl band that are indicative of different phases in the test sample;
searching for correlations between the C—H doublet bands and the components of the carbonyl band; and
changing a composition or processing conditions of a development sample of PHA based on presence or absence of the correlations to alter a crystallographic property of the development sample in an attempt to produce non-centrosymmetric β crystallographic structure in the development sample with stable piezoelectric properties.

2. The method of claim 1 further comprising preparing the test sample for spectroscopic examination.

3. The method of claim 2, wherein the preparing includes mounting the test sample on a stainless-steel slide.

4. The method of claim 1, wherein the monochromatic laser light is polarized monochromatic laser light.

5. The method of claim 1, wherein the PHA is polyhydroxybutyrate-hydroxyhexanoate (PHBHx).

6. The method of claim 5, wherein the changing includes changing a ratio of polyhydroxybutyrate (PHB) and hydroxyhexanoate (HHx).
